(19) United States
(12) Patent Application Publication
(10) Pub. No.: US 2015/0045628 A1
(43) Pub. Date: Feb. 12, 2015

(54) MULTI-LAYER PATCH FOR WIRELESS SENSOR DEVICES

(71) Applicant: Vital Connect, Inc., Campbell, CA (US)

(72) Inventors: Rod MOGHADAM, Hayward, CA (US); Azin SAHABI, San Francisco, CA (US); Stephen ZADIG, Portola Valley, CA (US)

(73) Assignee: Vital Connect, Inc., Campbell, CA (US)

(21) Appl. No.: 13/963,961

(22) Filed: Aug. 9, 2013

(51) Int. Cl. A61B 5/00

(52) U.S. Cl. CPC A61B 5/6833
USPC 600/300

(57) ABSTRACT

A wireless sensor device is disclosed. In a first aspect, the wireless sensor device comprises a housing unit and a patch coupled to the housing unit, wherein the patch includes a removable adhesive and is repositionable. In a second aspect, the wireless sensor device comprises a housing unit and a patch coupled to the housing unit, wherein the patch includes a plurality of removable adhesive layers that are each composed of removable medical grade silicone pressure sensitive adhesive (PSA).

### FIELD OF THE INVENTION

The present invention relates to wireless sensor devices, and more particularly, to a multi-layer patch for the wireless sensor devices.

### BACKGROUND

Wireless sensor devices are used in a variety of applications including the health monitoring of patients. In many of these applications, a wireless sensor device is attached directly to the user's skin (e.g. near the chest area) to measure certain physiological data. Conventional wireless sensor devices include attaching mechanisms that are cumbersome and utilize certain adhesives that cause irritation to the user's skin. Therefore, there is a strong need for a cost-effective solution that overcomes the aforementioned issues. The present invention addresses such a need.

### SUMMARY OF THE INVENTION

A wireless sensor device is disclosed. In a first aspect, the wireless sensor device comprises a housing unit and a patch coupled to the housing unit, wherein the patch includes a removable adhesive.

In a second aspect, the wireless sensor device comprises a housing unit and a patch coupled to the housing unit, wherein the patch includes a plurality of removable adhesive layers that are each composed of removable medical grade silicone pressure sensitive adhesive (PSA).

In a third aspect, the wireless sensor device comprises a housing unit that includes a sensor, a processor coupled to the sensor, a memory device coupled to the processor, wherein the memory device includes an application that is executable by the processor, and a transmitter coupled to the application, and a patch coupled to the housing unit, wherein the patch includes a plurality of removable adhesive layers that are each composed of removable medical grade silicone PSA.

### BRIEF DESCRIPTION OF THE DRAWINGS

The accompanying figures illustrate several embodiments of the invention and, together with the description, serve to explain the principles of the invention. One of ordinary skill in the art readily recognizes that the embodiments illustrated in the figures are merely exemplary, and are not intended to limit the scope of the present invention.

FIG. 1 illustrates a wireless sensor device in accordance with an embodiment.

FIG. 2 illustrates a wireless sensor device in a patch form factor in accordance with an embodiment.

FIG. 3 illustrates a more detailed view of a patch platform of the wireless sensor device in accordance with an embodiment.

### DETAILED DESCRIPTION

The present invention relates to wireless sensor devices, and more particularly, to a multi-layer patch for the wireless sensor devices. The following description is presented to enable one of ordinary skill in the art to make and use the invention and is provided in the context of a patent application and its requirements. Various modifications to the preferred embodiment and the generic principles and features described herein will be readily apparent to those skilled in the art. Thus, the present invention is not intended to be limited to the embodiments shown but is to be accorded the widest scope consistent with the principles and features described herein.

Attaching a wireless sensor device to a user enables the accurate measurement and monitoring of various health related information and biometrics. If the attachment is uncomfortable, the user may not be able to tolerate the wireless sensor device for prolonged periods of time. Accordingly, a system and method in accordance with the present invention develops a wireless sensor device in a patch form factor with an attachment mechanism that results in a comfortable experience to the patients without jeopardizing the performance and the life expectancy of the wireless sensor device.

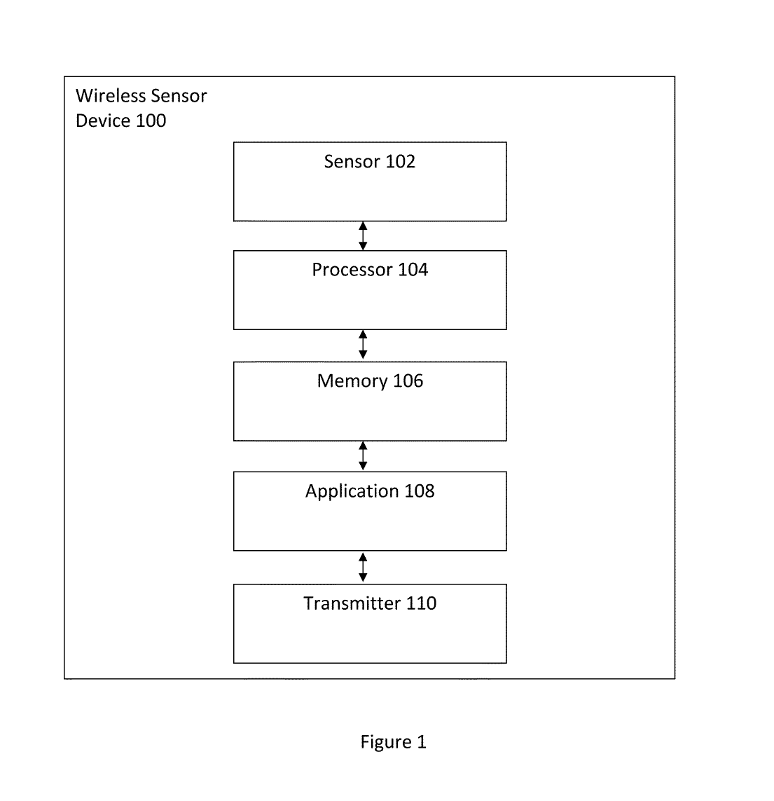

FIG. 1 illustrates a wireless sensor device 100 in accordance with an embodiment. The wireless sensor device 100 includes a sensor 102, a processor 104 coupled to the sensor 102, a memory 106 coupled to the processor 104, an application 108 coupled to the memory 106, and a transmitter 110 coupled to the application 108. In one embodiment, the wireless sensor device 100 is attached to a user via a multi-layer patch. The sensor 102 obtains data from the user and the data is then transmitted to the memory 106 via the processor 104 and in turn to the application 108. The processor 104 executes the application 108 to monitor information regarding the user's health. The information is transmitted to the transmitter 110 and in turn relayed to another user or device.

In one embodiment, the sensor 102 is a microelectromechanical system (MEMS) tri-axial accelerometer and the processor 104 is a microprocessor. One of ordinary skill in the art readily recognizes that a variety of devices can be utilized for the sensor 102, the processor 104, the memory 106, the application 108, and the transmitter 110 and that would be within the spirit and scope of the present invention.

One of ordinary skill in the art readily recognizes that a variety of wireless sensor devices can be utilized in conjunction with the multi-layer patch including but not limited to a wireless sensor device in a patch form-factor, tri-axial accelerometers, uni-axial accelerometers, bi-axial accelerometers, gyroscopes, and pressure sensors and that would be within the spirit and scope of the present invention.

To describe the features of the present invention in more detail, refer now to the following description in conjunction with the accompanying Figures.

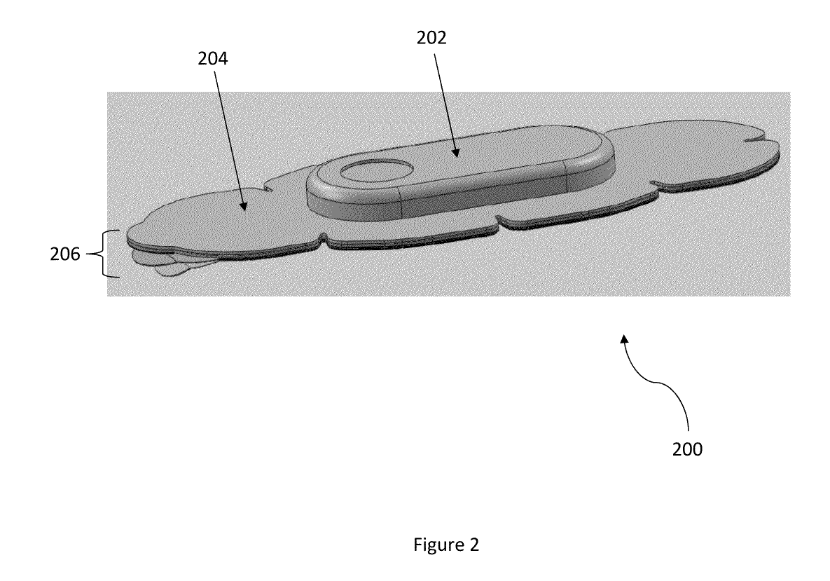

FIG. 2 illustrates a wireless sensor device 200 in a patch form factor in accordance with an embodiment. The wireless sensor device 200 includes a housing unit 202 coupled to a patch 204. In one embodiment, the patch 204 is one continuous unit with the housing unit 202. The housing unit 202 includes any of the sensor 102, the process 104, the memory 106, the application 108, and the transmitter 110. The patch 204 includes a plurality of removable adhesive layers 206.

In one embodiment, the plurality of removable adhesive layers 206 comprises three removable adhesive layers. In another embodiment, the plurality of removable adhesive layers 206 comprises five removable adhesive layers. In one embodiment, the removable adhesive is a removable medical grade silicone pressure sensitive adhesive (PSA).

In another embodiment, the silicone PSA is coated on a thermoplastic elastomer (TPE) carrier. Silicone PSA is a low trauma adhesive and does not damage dry and sensitive skin during the removal process of the wireless sensor device 200 from the user's skin. In one embodiment, each of the plurality of silicone PSA layers of the patch 204 is supported by a layer of medical grade polyethylene (PE) film for added tensile strength and structure. Therefore, in this embodiment, each of the plurality of silicone PSA layers of the patch 204 is laminated to a layer of PE film. In another embodiment, the layer of PE film is 0.0015″ thick.

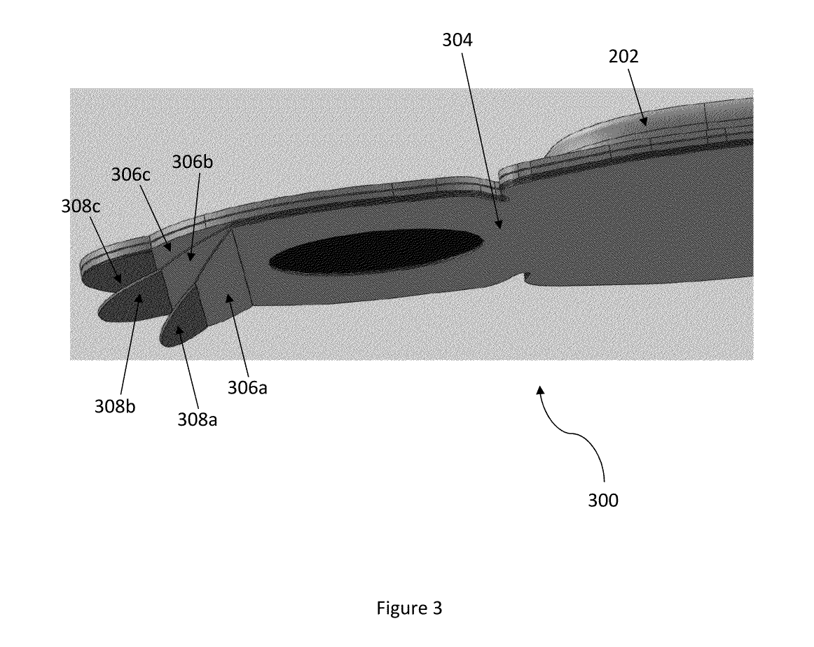

FIG. 3 illustrates a more detailed view 300 of a patch platform of the wireless sensor device 200 in accordance with an embodiment. In FIG. 3, the patch platform acts as a vehicle or place on which a sensor of the wireless sensor device 200 is mounted prior to attachment to a user. As aforementioned, the wireless sensor device 200 includes a housing unit 202 coupled to a patch platform 304. The patch platform 304 includes three removable adhesive layers 306a, 306b, and 306c. The three removable adhesive layers 306a, 306b, and 306c each have a corresponding tab 308a, 308b, and 308c positioned on the end of the patch platform 304 for easy access, removal and usage by the user of the wireless sensor device 200. In one embodiment, each corresponding tab 308a-c is made of a non-woven membrane with moderate tensile strength.

Each of the three removable adhesive layers 306a-c provides adherence to the user's skin for a predetermined time period. In one embodiment, the predetermined time period is 24 hours to minimize a high degree of adhesion between the wireless sensor device 200 and the user's skin. In another embodiment, the predetermined time period is 48 hours. After each predetermined time period interval, the patch platform 304 of the wireless sensor device 200 is removed from the user's skin and one of the three removable adhesive layers 306a-c is removed/peeled off to expose a new adhesive layer. Accordingly, in one embodiment, layer 306a is removed to expose new adhesive layer 306b and layer 306b is removed to expose new adhesive layer 306c.

In one embodiment, the three removable adhesive layers 306a-c are composed of a silicone adhesive that is gentle on the user's skin and does not cause irritation or discomfort while in contact with the user's skin. In one embodiment, the patch platform 304 of the wireless sensor device 200 is repositioned on the user's skin each time one of the three layers 306a-c is removed to add comfort. In another embodiment, the repositioning of the patch platform 304 occurs during the predetermined time period.

As above described, the system provides a wireless sensor device with a repositionable multi-layer patch platform to cater to a wide range of patients in different age brackets and with different skin sensitivity levels. The multi-layer patch platform adheres to the user's skin for a minimum of 3 days and minimizes the adverse reactions of the user's skin resulting from more aggressive attachment mechanisms (e.g. acrylic adhesives). Additionally, the multi-layer patch platform can be removed from the user's skin without causing skin trauma during the removal process.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. A wireless sensor device, comprising:
a housing unit; and
a patch coupled to the housing unit, wherein the patch includes a removable adhesive and is repositionable.

2. The wireless sensor device of claim 1, wherein the removable adhesive is a removable medical grade silicone pressure sensitive adhesive (PSA).

3. The wireless sensor device of claim 1, wherein the housing unit further comprises:
a sensor;
a processor coupled to the sensor;
a memory device coupled to the processor, wherein the memory device includes an application that is executable by the processor; and
a transmitter coupled to the application.

4. The wireless sensor device of claim 1, wherein the patch further comprises:
a plurality of removable adhesive layers, wherein each of the plurality of removable adhesive layers is supported by a film for added tensile strength and structure.

5. The wireless sensor device of claim 4, wherein the film is a layer of medical grade polyethylene.

6. The wireless sensor device of claim 4, wherein each of the plurality of removable adhesive layers includes a corresponding selectable tab.

7. The wireless sensor device of claim 6, wherein each corresponding tab is located on the end of the patch for easy access and usage.

8. The wireless sensor device of claim 1, wherein the patch is one continuous unit with the housing unit.

9. The wireless sensor device of claim 1, wherein the patch is repositionable after each of the plurality of removable adhesive layers is removed.

10. The wireless sensor device of claim 4, wherein the plurality of removable adhesive layers adheres to a user's skin for a predetermined time period.

11. The wireless sensor device of claim 10, wherein the predetermined time period is 24 hours.

12. A wireless sensor device, comprising:
a housing unit; and
a patch coupled to the housing unit, wherein the patch includes a plurality of removable adhesive layers that are each composed of removable medical grade silicone pressure sensitive adhesive (PSA).

13. The wireless sensor device of claim 12, wherein the housing unit further comprises:
a sensor;
a processor coupled to the sensor;
a memory device coupled to the processor, wherein the memory device includes an application that is executable by the processor; and
a transmitter coupled to the application.

14. The wireless sensor device of claim 12, wherein each of the plurality of removable adhesive layers is supported by a film for added tensile strength and structure.

15. The wireless sensor device of claim 14, wherein the film is a layer of medical grade polyethylene.

16. The wireless sensor device of claim 12, wherein each of the plurality of removable adhesive layers includes a corresponding selectable tab.

17. The wireless sensor device of claim 12, wherein the patch is repositionable.

18. The wireless sensor device of claim 17, wherein the patch is repositionable after each of the plurality of removable adhesive layers is removed.

19. The wireless sensor device of claim 12, wherein the plurality of removable adhesive layers adheres to a user's skin for a predetermined time period.

20. A wireless sensor device, comprising:
a housing unit that includes a sensor, a processor coupled to the sensor, a memory device coupled to the processor, wherein the memory device includes an application that is executable by the processor, and a transmitter coupled to the application; and
a patch coupled to the housing unit, wherein the patch includes a plurality of removable adhesive layers composed of removable medical grade silicone pressure sensitive adhesive (PSA).
